(19) United States
(12) Patent Application Publication
(10) Pub. No.: US 2026/0239510 A1
(43) Pub. Date: Aug. 13, 2026

(54) LIGHTING CONTROL BASED ON SWEAT SENSING TECHNICAL FIELD

(71) Applicant: SIGNIFY HOLDING B.V., EINDHOVEN (NL)

(72) Inventors: TIES VAN BOMMEL, HORST (NL); PETER DEIXLER, ARLINGTON, MA (US)

(21) Appl. No.: 19/149,660

(22) PCT Filed: Jan. 16, 2024
(86) PCT No.: PCT/EP2024/050901

§ 371 Date: Jul. 21, 2025

Related U.S. Application Data
(60) Provisional application No. 63/441,000, filed on Jan. 25, 2023.

(30) Foreign Application Priority Data
Feb. 17, 2023 (EP) 23157267.8

(51) Int. Cl. H05B 47/105; H05B 45/10; H05B 45/20

(52) U.S. Cl. CPC H05B 47/105; H05B 45/10; H05B 45/20

(57) ABSTRACT

A lighting control method includes receiving sweat data from a sensor that is in proximity of a person in an area. The sensor is configured to sense sweat produced by the person. The method further includes processing the sweat data to determine one or more parameters related to one or more sweat components of the sweat and adjusting a spectral distribution of an illumination light at least based on the one or more parameters, where the illumination light is provided by one or more lighting devices that are in the area.

### BACKGROUND

The present disclosure relates generally to lighting control, and more particularly to lighting control based on sweat sensing.

In some cases, a person's physiological and psychological state may be affected by lighting. To improve a person's health by changing the lighting, it may be desirable to first determine the person's physiological and psychological state. In some cases, sweat may carry information that is indicative of a person's physiological and psychological state. Because sweat is generally readily accessible at the skin surface of a person, sensing sweat may offer a non-intrusive way of obtaining information that can be used to check and improve the physiological and psychological state of a person. Thus, a solution that enables lighting control based on information obtained from sweat to improve a person's health may be desirable.

### SUMMARY

The present disclosure relates generally to lighting control, and more particularly to lighting control based on sweat sensing. In an example embodiment, a lighting control method includes receiving sweat data from a sensor attached to a person in an area. The sensor is configured to sense sweat produced by the person. The method further includes processing the sweat data to determine one or more parameters related to one or more sweat components of the sweat and adjusting a spectral distribution of an illumination light at least based on the one or more parameters, where the illumination light is provided by one or more lighting devices that are in the area.

In another example embodiment, a lighting control system includes a controller configured to receive sweat data from a sensor attached to a person in an area. The sensor is configured to sense sweat produced by the person. The controller is further configured to process the sweat data to determine one or more parameters related to one or more sweat components of the sweat. The controller is also configured to adjust a spectral distribution of an illumination light at least based on the one or more parameters, where the illumination light is provided by one or more lighting devices that are in the area.

These and other aspects, objects, features, and embodiments will be apparent from the following description and the appended claims.

### BRIEF DESCRIPTION OF THE FIGURES

Reference will now be made to the accompanying drawings, which are not necessarily drawn to scale, and wherein:

FIG. 1 illustrates a lighting system that controls lighting based on sweat data according to an example embodiment;

FIG. 2 illustrates a spectral distribution of a light provided by the lighting system according to an example embodiment;

FIG. 3 illustrates a method of lighting control based on sweat data from a sensor according to an example embodiment; and

FIG. 4 illustrates a method of lighting control based on sweat data from a light fixture integrated sensor according to another example embodiment.

The drawings illustrate only example embodiments and are therefore not to be considered limiting in scope. The elements and features shown in the drawings are not necessarily to scale, emphasis instead being placed upon clearly illustrating the principles of the example embodiments. Additionally, certain dimensions or placements may be exaggerated to help visually convey such principles. In the drawings, the same reference numerals used in different drawings may designate like or corresponding but not necessarily identical elements.

### DETAILED DESCRIPTION OF THE EXAMPLE EMBODIMENTS

In the following paragraphs, example embodiments will be described in further detail with reference to the figures. In the description, well known components, methods, and/or processing techniques are omitted or briefly described. Furthermore, reference to various feature(s) of the embodiments is not to suggest that all embodiments must include the referenced feature(s).

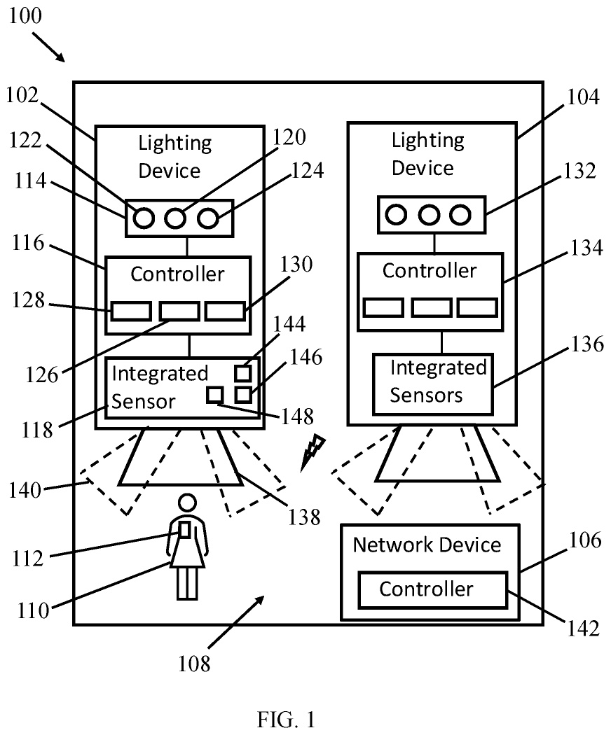

FIG. 1 illustrates a lighting system that controls lighting based on sweat data according to an example embodiment. In some example embodiments, the system 100 may include lighting devices 102, 104 (e.g., light fixtures). The lighting devices 102, 104 may be located in an area 108. For example, the area 108 may be a room or a hallway. A person 110 may be in the area 108. A sensor 112 may be in a proximity of the person 110 (e.g., attached to the person 110 as shown in FIG. 1 or in the lighting device 102 or another device in the area 108). For example, the sensor 112 may include one or more sweat sensors that sense sweat produced by the person 110. For example, the sensor 112 may sense sweat continuously or at regular intervals. The sensor 112 may also include other sensors such as a body temperature sensor, a heart rate sensor, a respiration sensor, etc. The sensor 112 may be attached to the skin of the person 110, for example, by a tape or a band. The sensor 112 may be arranged in one or more electronic tattoos, bands, and/or patches. Alternatively, the sensor 112 may be built-in with a clothing of the person 110 such that sensor 112 can come in contact with the person's skin or otherwise positioned to sense the sweat produced by the person 110.

In some example embodiments, the lighting device 102 may include a light module 114 that may include light source units 120, 122, 124. The light source unit 120 may include multiple light emitting diodes (LEDs) that are controllable to emit an illumination light (i.e., an illumination white light). For example, the light source unit 120 may include LEDs that can emit lights that have different wavelengths. To illustrate, the light source unit 120 may include one or more LEDs that can emit a red light that has a wavelength in the general range of 600-720 nanometers (nm), one or more LEDs that can emit a green light that has a wavelength in the general range of 520-560 nm, one or more LEDs that can emit a blue light that has a wavelength in the general range of 420-480 nm, one or more LEDs that can emit a cyan light that has a wavelength in the general range of 480-520 nm, one or more LEDs that can emit a yellow light that has a wavelength in the general range of 560-580 nm, etc. By controlling the contribution of the individual lights provided by the LEDs of the light source unit 120, the light device 102 may provide an illumination light (i.e., an illumination white light) that has a desired intensity, correlated color temperature (CCT), and/or spatial distribution. For example, the CCT of the illumination light provided by the lighting device 102 may a range from 1800K to 6500K and may have color rendering index (CRI) of at least 70.

In some example embodiments, the light source unit 122 may include one or more LEDs that emit an infrared light. The LEDs of the light source unit 122 may emit an infrared light having one or more wavelength components in a range above 720 nm. For example, the infrared light provided by the light source unit 122 may be a near-infrared light. To illustrate, the near-infrared light provided by the light source unit 122 may be in a range between 720 nm and 1400 nm. The infrared light may alternatively have a wavelength above 1400 nm.

In some example embodiments, the light source unit 124 may include one or more LEDs that emit an ultraviolet (UV) light. The LEDs of the light source unit 124 may emit an UV light having one or more wavelength components in a range below 390 nm. For example, the UV light provided by the light source unit 124 may be a UV-C that may be effective in killing bacteria.

In some example embodiments, the lighting device 102 may also include a controller 116 that can control the operation of the lighting device 102. For example, the controller 116 may control the light module 114 to control the light provide by the lighting device 102. To illustrate, the controller 116 may control the intensity, CCT, the spectral distribution, and spatial distribution of the illumination light provide by the light source unit 120. As another example, the controller 116 may control whether the light provide provided by the light module 114 includes an infrared light and/or a UV-C light in addition to the illumination light provided by the light source unit 120.

In some example embodiments, the controller 116 may include a microprocessor 126, a memory device 128 (e.g., a flash memory unit), and a communication unit 130. The memory device 128 may store software code that is executable by the microprocessor 126 to perform operations described herein with respect to the controller 116. The memory device 128 may also store data that is used in or generated by the execution of the executable software code. The controller 116 may use the communication unit 130 to transmit and received data (e.g., sensor data, lighting information, and lighting command) wirelessly (e.g., Wi-Fi, BLE, ZigBee, UWB, and/or cellular network) and/or via one or more wired connections (e.g., Ethernet).

In some example embodiments, the lighting device 102 may include an integrated sensor 118 that may include one or more sensors units. The integrated sensor 118 may provide sensor data to the controller 116 that can control the light provided by the lighting device 102 and the light provided by the lighting device 104 based on the sensor data. To illustrate, the integrated sensor 118 may include a location sensor unit 144, an activity sensor unit 146, a volatile organic compounds (VOC) sensor unit 148, and/or other sensor units. For example, the integrated sensor 118 may include a thermopile sensor, a passive infrared sensor, and/or another sensor that may be used as the location sensor unit 144 to detect whether the area 108 is occupied and determine the location of the person 110 in the area 108.

In some example embodiments, the integrated sensor 118 may also include microphones, a thermopile sensor, a humidity sensor, temperature sensor, and/or another sensor that operate individually or together as the activity sensor unit 146 to determine the activity of the person 110. For example, the activity sensor unit 146 may be used to determine whether the person 110 has just finished exercising based on the breathing rate (i.e., respiratory rate) of the person 110, sounds emitted by the person 110, the blood pressure of the person 110, etc. As another example, the activity sensor unit 146 may be used to determine whether the person 110 has just showered based on the humidity in the air and/or sounds associated with showering. As another example, the activity sensor unit 146 may be used to determine whether the person 110 has used a deodorant based on the sound associated with the spraying of a deodorant. The VOC sensor unit 148 may detect compounds, such as cortisol, in the air that may be in the sweat produced by the person 110 or that may result from bacteria that act on the sweat produced by the person 110. For example, the VOC sensor unit 148 may be an electronic nose (e-nose) device that may include an array of sensors that sense substances (e.g., human-emitted VOCs) in the air. For example, based on the detection of substances in the air in the area 108, sensor data from the VOC sensor unit 148 may indicate or may be used to determine the amount or concentration of cortisol and/or other components of the sweat produced by the person 110.

In some example embodiments, the lighting device 104 may include a light module 132, a controller 134, and an integrated sensor 136. The light module 132 may include different light source units that corresponds to the light source units 120, 124, 126 of the lighting device 102. The controller 134 may include a microprocessor, a memory device, and a communication unit that correspond to the microprocessor 126, the memory device 128, and the communication unit 130 of the controller 116 of the lighting device 102. The integrated sensor 136 may correspond to the integrated sensor 118 of the lighting device 102. In general, the lighting device 104 may operate in the same manner as described with respect to the lighting device 102.

In some example embodiments, the sensor 112 may be attached to the person 110 and may sense one or more substances (i.e., sweat components/biomarkers) in sweat produced by the person 110. For example, the sensor 112 may detect one or more ions or compounds (i.e., analytes) that may be in the sweat produced by the person 110. The sensor 112 may detect the presence of analytes and the amounts and/or concentrations of the analytes in sweat electrically and/or optically as can be readily understood by those of ordinary skill in the art with the benefit of this disclosure. For example, the sensor 112 may detect the presence, amount, and/or concentration of lactate in sweat produced by the person 110. As another example, the sensor 112 may detect the presence, amount, and/or concentration, of cortisol in sweat produced by the person 110. As another example, the sensor 112 may detect the presence, amount, and/or concentration in sweat produced by the person 110. As another example, the sensor 112 may detect the presence, amount, and/or concentration of sodium, potassium, and/or chloride in sweat produced by the person 110.

In some example embodiments, the sensor 112 may transmit sensor data wirelessly (e.g., Wi-Fi, BLE, ZigBee, UWB, and/or cellular network). The sensor data may include information sensed/detected, measured, and/or determined by the sensor 112. For example, the sensor data may include information related to one or more of sweat produced by the person 110, the body temperature of the person 110, the heart rate of the person 110, respiratory rate of the person 110, etc. The sensor 112 may transmit the sensor data to the lighting device 102, to the lighting device 104, and/or to a network device 106 (e.g., a server).

In some example embodiments, the controller 116 of the lighting device 102 may receive the sensor data from the sensor 112 and process the sensor data. The controller 116 may control (e.g., adjust, turn off, turn on) the light provided by the lighting device 102 based on the sensor data. To illustrate, the microprocessor 126 of the controller 116 may receive the sensor data via the communication unit 130 of the controller 116 and process the sensor data. The microprocessor 126 may control the light module 114 to adjust the light provided by the light module 114 based on the sensor data. For example, the sensor 112 may sense sweat continuously or at regular intervals and send the sensor data to the lighting device 102, and the microprocessor 126 may control the light module 114 to adjust the illumination light provided by the lighting device 102 (e.g., change the spectral distribution of the light) continuously or at regular intervals (e.g., every 1 minute, every 10 minutes, every 30 minutes, every hour).

In some example embodiments, the sensor data may include sweat data that indicates the amount or concentration of sodium and/or potassium in sweat produced by the person 110. Alternatively or in addition, the sensor data may include sweat data that may be used to determine the amount or concentration of sodium and/or potassium in sweat produced by the person 110. The microprocessor 126 may receive and process the sensor data to extract or otherwise determine one or more parameters related to one or more substances (i.e., sweat components/biomarkers) in the sweat, such as the amount or concentration of sodium and/or potassium in the sweat of the person 110. The microprocessor 126 may determine from the parameters whether the person 110 is dehydrated or well hydrated. For example, because the content of sodium and potassium in sweat decreases when a person is dehydrated, such information may be used to determine whether a person is dehydrated or well hydrated. As another example, because the electrical conductivity of sweat varies in proportion to amount of sodium in the sweat, such information may be used to indirectly determine whether a person is dehydrated or well hydrated.

If the microprocessor 126 determines that the person 110 is dehydrated, the microprocessor 126 may control the light module 114 to reduce or turn off an infrared light component that may be included in the light provided by the light module 114. For example, the microprocessor 126 may control the light module 114 such that infrared light that may have been emitted by the light source unit 122 of the light module 114 is turned off or reduced in intensity. Turning off or reducing the infrared light component of the light provided by the light module 114 changes spectral distribution of the light provided by the light module 114 although the spectral distribution of the illumination light emitted by the light source unit 120 is unchanged. Because the spectral distribution of the illumination light emitted by the light source unit 120 is unchanged, the CCT of the light provided by the lighting device 102 remains the same. In general, the spectral distribution of a light refers to the distribution of light energy at different wavelengths of the light as can be readily understood by those of ordinary skill in the art. For example, a graph of a spectral distribution of a light may show intensities of the light at different wavelengths.

In some example embodiments, if the microprocessor 126 determines that the person 110 is dehydrated, the microprocessor 126 may control the light module 114 to reduce the intensity of the light provided by the light module 114. For example, the microprocessor 126 may control the light module 114 to change the spectral distribution of the light provided by the light module 114 by reducing the intensity of one or more light components of the light provided by the light module 114. To illustrate, the microprocessor 126 may control the light module 114 to reduce the intensities of blue light (e.g., 455-490 nm wavelength range) and red light (e.g., 625-720 nm wavelength range) in the light provided by the light module 114. Although the spectral distribution of the light provided by the light module 114 is changed, by adjusting intensities of blue light and red light that have counteracting effect on CCT, the CCT of the light may be generally maintained constant or within a range such that the change (e.g., 300K) in the CCT of the light provided by the light module 114 is imperceptible by the person 110.

In some example embodiments, if the microprocessor 126 determines that the person 110 is dehydrated, the microprocessor 126 may control the light module 114 and transmit lighting commands to other lighting devices in the area 108 to provide a guiding light, for example, toward an exit. If the microprocessor 126 determines based on the sensor data that the person 110 is well hydrated, the microprocessor 126 may control the light module 114 to include infrared (e.g., near-infrared light) in the light provided by the light module 114 or to increase the intensity of the infrared (e.g., near-infrared light) in the light. For example, the microprocessor 126 may control the light module 114 such that the light source unit 122 starts emitting the infrared light (e.g., near-infrared light) or increases the intensity of the infrared light (e.g., near-infrared light) already emitted by the light source unit 122.

In some example embodiments, the controller 116 may extract or otherwise determine from the sensor data one or more parameters such as the ratio of analytes in the sweat of the person 110 and control the light provided by the lighting device 102 based on the parameters. For example, the controller 116 may determine the ratio of sodium and potassium, the ratio of chloride to sodium, etc. The microprocessor 126 may determine, for example, whether the person 110 is dehydrated based on one or more ratios of analytes and control the light provided by the light module 114 accordingly in the manner described above. The microprocessor 126 may also determine other information indicating of the physiological and/or psychological state (e.g., stress level) of the person based on the ratio of analytes and control the light provided by the light module 114 accordingly in the manner described above.

In some example embodiments, the sensor data may include information that indicates the amount or concentration of glucose in sweat produced by the person 110. Alternatively or in addition, the sensor data may include information that may be used to determine the amount or concentration of glucose in sweat produced by the person 110. The microprocessor 126 may receive and process the sensor data to extract or otherwise determine one or more parameters such as the amount or concentration of glucose in the sweat of the person 110. The microprocessor 126 may determine from the parameters whether the glucose level of the person 110 is a normal, high, or low.

If the microprocessor 126 determines that the glucose level of the person 110 is low or below a glucose threshold, the microprocessor 126 may control the light module 114 to emit a light that may help facilitate the production of more cortisol by the person 110. For example, the glucose threshold can be set to a desired value that is specific to the person 110 or that is generally recommended for the population, for example, by a governmental or private health entity. The microprocessor 126 may control the light module 114 such that the light source unit 120 emits cyan light (e.g., approximately 500 nm wavelength light) or increases the intensity of the cyan light in the light provided by the light module 114. The introduction of the cyan light or the increase in the intensity of the cyan light may change the spectral distribution of the light provided by the lighting device 102.

In some example embodiments, the microprocessor 126 may also control the light module 114 to maintain the CCT of the light provided by the light module 114 constant or within a range such that the change (e.g., 300K) in the CCT of the light resulting from the change in the cyan light is imperceptible by the person 110. For example, the microprocessor 126 may control the light module 114 to introduce a yellow light (e.g., approximately 590 nm wavelength light) to counteract the effect of the cyan light.

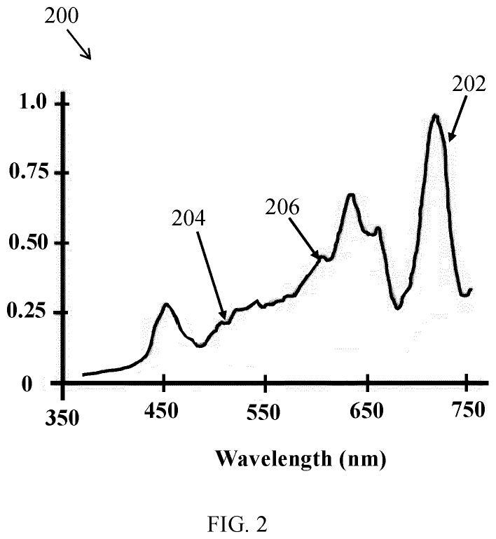

FIG. 2 illustrates a graph 200 of a spectral distribution 202 of the light provided by the lighting device 102 according to an example embodiment. In FIG. 2, the wavelengths of the light are represented on the horizontal axis, and normalized amplitude values are represented on the vertical axis. For example, a cyan light having a wavelength around 500 nm as shown by the arrow 204 may be introduced to the light provided by the lighting device 102 and may result in a change in the spectral distribution 202 of the light. Alternatively, the intensity of a cyan light that is already a component of the light provided by the lighting device 102 may be increased resulting in a change in the spectral distribution 202 of the light provided by the lighting device 102. A yellow light having a wavelength around 590 nm as shown by the arrow 206 may be introduced to the light provided by the lighting device 102 and may result in a change in the spectral distribution 202 of the light. Alternatively, the intensity of a yellow light that is already a component of the light provided by the lighting device 102 may be increased resulting in a change in the spectral distribution 202 of the light provided by the lighting device 102. As described above, the yellow light may be introduced or the intensity of the yellow light may be increased to counteract CCT change resulting from the introduction of the cyan light.

Referring again to FIG. 1, the microprocessor 126 determines that the glucose level of the person 110 is high, the microprocessor 126 may control the light module 114 to turn off or reduce the intensity of a light component of the light provided by the light module 114 to help reduce the production of cortisol by the person 110. For example, the microprocessor 126 may control the light module 114 to turn off cyan light emitted by the light source unit 120. Alternatively, the microprocessor 126 may control the light module 114 to reduce the intensity of the cyan light emitted by emitted by the light source unit 120. Turning off or reducing the intensity of the cyan light changes the spectral distribution of the light provided by the lighting device 102. To maintain the CCT of the light provided by the lighting device 102 within a range such that the change in the CCT of the light is imperceptible by the person 110, the microprocessor 126 may control the light module 114 to turn off or reduce a light component (e.g., yellow light) emitted by the light source unit 120 to counteract the impact of the change in in the cyan light.

In some example embodiments, if the microprocessor 126 determines that the glucose level of the person 110 is high, the microprocessor 126 may control the light module 114 such that the light source unit 124 emits UV-B light (e.g., 310 nm UV light) to help reduce the production of glucose by the person 110. Introducing or increasing the UV light component of the light provided by the light module 114 changes spectral distribution of the light provided by the light module 114 although the spectral distribution of the illumination light emitted by the light source unit 120 is unchanged. Because the spectral distribution of the illumination light emitted by the light source unit 120 is unchanged by the change in the UV light, the CCT of the light provided by the lighting device 102 can be maintained.

In some example embodiments, the sensor data may include information that indicates the amount or concentration of lactate in sweat produced by the person 110. Alternatively or in addition, the sensor data may include information that may be used to determine the amount or concentration of lactate in sweat produced by the person 110. The microprocessor 126 may receive and process the sensor data to extract or otherwise determine one or more parameters such as the amount or concentration of lactate in the sweat of the person 110. The microprocessor 126 may determine from the parameters whether the lactate level or concentration is a normal, high, or low. If the microprocessor 126 determines that the lactate level or concentration is too high or exceeds a lactate threshold (which may indicate inflammation), the microprocessor 126 may control the light module 114 to emit a near-infrared light (e.g., 720-1000 nm wavelength) or increase the intensity of near-infrared light in the light provided by the light module 114. For example, the lactate threshold can be set to a desired value that is specific to the person 110 or that is generally recommended for the general population, for example, by a governmental or private health entity. Turning on or increasing the infrared light emitted by the light source unit 122 changes the spectral distribution of the light provided by the light module 114 while the spectral distribution of the illumination light emitted by the light source unit 120 remains unchanged. Because the spectral distribution of the illumination light emitted by the light source unit 120 is unchanged, the CCT of the light provided by the lighting device 102 is the same before and after the introducing or increase of the intensity the infrared light.

In some example embodiments, the sensor data may include information that indicates the amount or concentration of cortisol in sweat produced by the person 110. Alternatively or in addition, the sensor data may include information that may be used to determine the amount or concentration of cortisol in sweat produced by the person 110. The microprocessor 126 may receive and process the sensor data to extract or otherwise determine one or more parameters such as the amount or concentration of cortisol in the sweat of the person 110. If the microprocessor 126 determines that the cortisol level or concentration is low or below a low cortisol threshold, the microprocessor 126 may control the light module 114 to emit a light that may help facilitate the production of more cortisol by the person 110. For example, the low cortisol threshold can be set to a desired value that is specific to the person 110 (e.g., based on a recommendation of a medical professional) or that is generally recommended for the general population, for example, by a governmental or private health entity. To help increase cortisol production by the person 110, the microprocessor 126 may control the light module 114 to introduce cyan light (e.g., approximately 500 nm wavelength light) or increase the intensity of the cyan light in the light provided by the light module 114. The introduction of the cyan light or the increase in the intensity of the cyan light may change the spectral distribution of the light provided by the lighting device 102.

In some example embodiments, the microprocessor 126 may also control the light module 114 to maintain the CCT of the light provided by the light module 114 constant or within a range such that change (e.g., 300K) in the CCT of the light resulting from the change in the cyan light is imperceptible by the person 110. For example, the microprocessor 126 may control the light module 114 to introduce a yellow light (e.g., approximately 590 nm wavelength light) to counteract the effect of the cyan light.

If the microprocessor 126 determines that the cortisol level or concentration is high or above a high cortisol threshold (e.g., due to sleep apnea), the microprocessor 126 may control the light module 114 to reduce or turn off cyan light (e.g., approximately 500 nm wavelength light) in the light provided by the light module 114. For example, the high cortisol threshold can be set to a desired value that is specific to the person 110 or that is generally recommended for the general population, for example, by a governmental or private health entity. The reduction in the intensity of the cyan light or turning off the cyan light may change the spectral distribution of the light provided by the lighting device 102. To maintain the CCT of the light provided by the lighting device 102 within a range such that CCT change is imperceptible by the person 110, the microprocessor 126 may control the light module 114 to reduce the intensity of another light component (e.g., yellow light) of the light provided by the light module 114.

In some example embodiments, the sensor data may include information that indicates or that can be used to determine the rate of sweating (i.e., sweat rate) by the person 110. The microprocessor 126 may receive and process the sensor data to extract or otherwise determine one or more parameters such as the sweat rate of the person 110. Because more odor is produced by bacteria (e.g., Staphylococcus, Corynebacterium, and/or Propionibacterium) on the skin (e.g., scalp) of the person 110 at higher sweat rates, if the microprocessor 126 determines that the sweat rate of the person 110 exceeds a threshold (e.g., 2 liters per day), the microprocessor 126 may control the light module 114 to emit a light that may help kill some of the bacteria. For example, the microprocessor 126 may control the light module 114 such that the light source unit 120 starts emitting or increases the intensity of violet light (e.g., in a range of 390-455 nm wavelength). The violet light may change the spectral distribution of the light provided by the lighting device 102. To counteract the effect of the violet light on the CCT of the light provided by the light module 114, the microprocessor 126 may control the light module 114 such that the light source unit 120 emits, for example, orange light and/or red light. For example, the CCT of the light provided by the light module 114 may be maintained within a range (e.g., 300K) such that the change in CCT of the light provided by the lighting device 102 is imperceptible by the person 110.

In some example embodiments, if the microprocessor 126 determines that the sweat rate of the person 110 exceeds the threshold (e.g., 1 liter per day), the microprocessor 126 may control the light module 114 such that the light source unit 124 emits or increase the intensity of UV light to kill the bacteria on the skin (e.g., scalp) of the person 110.

Introducing or increasing the UV light component of the light provided by the light module 114 changes spectral distribution of the light provided by the light module 114 although the spectral distribution of the illumination light emitted by the light source unit 120 is unchanged. Because the spectral distribution of the illumination light emitted by the light source unit 120 is unchanged by the change in the UV light, the CCT of the light provided by the lighting device 102 can be maintained.

In some example embodiments, the sensor data may include information that indicates or that may be used to determine whether the sweat produced by the person 110 is regular sweat or stress sweat. Typically, regular sweat is produced by eccrine sweat glands of a person when the person's body is hot. Regular sweat typically includes water, sodium, and potassium. Stress sweat is produced by apocrine glands of a person as a result of an emotion such as anxiety, stress, or excitement. Stress sweat is generally milkier than regular sweat and includes fatty acids and proteins that are not typically in regular sweat.

In some example embodiments, the microprocessor 126 may receive and process the sensor data to extract or otherwise determine one or more parameters that indicate whether the sweat produced by the person 110 is regular sweat or stress sweat. If the microprocessor 126 determines that the sweat produced by the person 110 is stress sweat, the microprocessor 126 may control the light module 114 to emit the microprocessor 126 may control the light module 114 to reduce or turn off cyan light (e.g., approximately 500 nm wavelength light) in the light provided by the light module 114. The reduction in the intensity of the cyan light or turning off the cyan light may change the spectral distribution of the light provided by the lighting device 102. To maintain the CCT of the light provided by the lighting device 102 within a range such that CCT change is imperceptible by the person 110, the microprocessor 126 may control the light module 114 to reduce the intensity of another light component (e.g., yellow light) of the light provided by the light module 114. For example, the microprocessor 126 may control the light module 114 such that the change in the CCT of the light provided by the lighting device 102 is less than 300K.

In some example embodiments, the controller 116 of the lighting device 102 may control the spatial distribution of the light provided by the lighting device 102 based on one or more parameters extracted and/or determined from sensor data received from the sensor 112. The spatial distribution of a light generally refers to the direction, shape, width, and/or pattern of the light provided by one or more lighting devices as can be readily understood by those of ordinary skill in the art. The controller 116 may change the spatial distribution of the light provided by the lighting device 102 instead of or in addition to changing the spectral distribution of the light. To illustrate, in response to determining that the person 110 is dehydrated as described above, the controller 116 may control the light module 114 to change the spatial distribution of the light provided by the lighting device 102 from a first spatial distribution 138 to a second spatial distribution 140 shown in FIG. 1 or to another spatial distribution. Alternatively, the controller 116 may control the light module 114 to change the spatial distribution of the light provided by the lighting device 102 from the second spatial distribution 140 to the first spatial distribution 138 or to another spatial distribution. The controller 116 may control the orientation of the light module 114 or turn on some LEDs and turn off other LEDs of light source unit 120 of the light module 114 to change the spatial distribution of the light provided by the lighting device 102.

In some example embodiments, the lighting device 102 may operate as a master lighting device, where the controller 116 controls operations of other lighting devices including the lighting device 104 in the lighting system 100 to control the light provided by the lighting system 100. To illustrate, the controller 116 of the lighting device 102 may control the light provided by the lighting device 102 as well as the light provided by the lighting device 104 based on one or more parameters extracted and/or determined from sensor data received from the sensor 112. For example, the controller 116 may transmit a lighting control command generated based on the one or more parameters to the lighting device 104, and the controller 134 of the lighting device 104 may receive the lighting control command and control the light provided by the light module 132 of the lighting device 104 accordingly.

In some example embodiments, the controller 116 of the lighting device 102 may control other lighting devices of the lighting system 100 including the lighting device 104 to control the spatial distribution of the light provided by the lighting system 100. For example, the controller 116 may determine based on the received sensor data that the person 110 is stressed. In response, the controller 116 may control the light module 114 and transmit lighting control commands to the lighting device 104 to adjust the spatial distribution of the light provided by each lighting device of the lighting devices 102, 104. To illustrate, the controller 116 may control the light module 114 of the lighting device 102 such that the light provided by the lighting device 102 is dimmer than the light provided by the lighting device 104, thereby changing the spatial distribution of the light provided by the lighting system 100. Alternatively, the controller 116 may control the light module 132 of the lighting device 104 such that the light provided by the lighting device 104 is dimmer than the light provided by the lighting device 102, thereby changing the spatial distribution of the light provided by the lighting system 100.

In some example embodiments, the sensor 112 may include a temperature sensor that measures the temperature of the person 110. The sensor 112 may also include a heart rate sensor that measures the heart rate of the person 110. The sensor 112 may also include a respiration sensor that measures the respiratory rate of the person 110. The sensor data transmitted by the sensor 112 may indicate the temperature, the heart rate (e.g., absolute heart rate and/or heart rate variability), the blood pressure, and/or the respiratory rate of the person 110. The controller 116 may adjust the spectral distribution and the spatial distribution of the light provided by the lighting device 102 based on the temperature, the heart rate, the blood pressure, and/or the respiratory rate of the person 110 in addition to the sweat data received from the sensor 112. For example, a relatively higher body temperature and/or heart rate may provide additional indications that the person 110 is dehydrated, and the controller 116 may adjust the spectral distribution and/or the spatial distribution of the light provided by the lighting device 102 to alleviate the dehydration of the person 110 as described above.

In some example embodiments, the controller 116 may adjust the spectral distribution and the spatial distribution of the light provided by the lighting device 102 and/or the lighting device 104 based on sensor data from the integrated sensor 118 in the same manner as described with respect to sensor data from the sensor 112. As described above, the sensor data from the integrated sensor 118 may include information from the location sensor unit 144, the activity sensor unit 146, and/or the VOC sensor unit 148. The controller 116 may adjust the spectral distribution and/or the spatial distribution of the light provided by the lighting devices 102, 104 based on one or more parameters extracted or otherwise determined from sweat data received from the VOC sensor unit 148. For example, the sensor data from the integrated sensor 118 may include sweat data from the VOC sensor unit 148 that may indicate or may be used to determine the amount or concentration of cortisol and/or other sweat components in sweat produced by the person 110.

In some example embodiments, the microprocessor 126 may control the VOC sensor unit 148 to perform sensing of compounds (e.g., cortisol) in the air after determining that the likelihood of reliably detecting sweat components of the sweat produced by the person 110 is good. Alternatively or in addition, before adjusting the light provided by the lighting devices 102, 104 based on sweat data from the VOC sensor unit 148, the microprocessor 126 of the controller 116 may determine whether the sweat data from the VOC sensor unit 148 is reliable based on information from the location sensor unit 144 and/or the activity sensor unit 146. For example, before processing and/or using the sweat data from the VOC sensor unit 148, the microprocessor 126 may determine whether the area 108 is occupied based on information from the location sensor unit 144. To illustrate, if the area 108 is occupied, the likelihood that the one or more compounds detected by the VOC sensor unit 148 are from sweat produced by the occupant (e.g., the person 110) is higher.

As another example, before processing and/or using the sweat data from the VOC sensor unit 148, the microprocessor 126 may determine whether the person 110 just finished exercising based on information from the activity sensor unit 146 (e.g., from a thermopile sensor). To illustrate, if the person 110 just finished exercising (e.g., based on heart rate and/or breathing rate), the likelihood that the one or more compounds detected by the VOC sensor unit 148 are from sweat produced by the person 110 is higher.

As yet another example, before processing and/or using the sweat data from the VOC sensor unit 148, the microprocessor 126 may determine whether the person 110 just showered and has not used a deodorant (which can affect the reliability of sensing by the VOC sensor unit 148) based on information from the activity sensor unit 146 (e.g., from a humidity sensor and microphones). To illustrate, if the person 110 just finished showering and has not yet used a deodorant, the likelihood that one or more sweat components detected by the VOC sensor unit 148 are from sweat produced by the person 110 instead of from the deodorant is relatively higher.

In some example embodiments, information from the location sensor unit 144 can be used in conjunction with information from the activity sensor unit 146 to determine the likelihood that the sweat data from the VOC sensor unit 148 is reliable. To illustrate, the controller 116 may determine the location of the person 110 with respect to the lighting device 102 before using the sweat data from the VOC sensor unit 148. The controller 116 may determine the location of the person 110 with respect to the lighting device 102 based on information from the location sensor unit 144 of the integrated sensor 118. For example, the controller 116 may determine the location of the person 110 based on IR energy detected by a thermopile sensor of the location sensor unit 144. Alternatively or in addition, the controller 116 may determine the location of the person 110 based on Wi-Fi sensing, sound processing, etc. as can be readily understood by those of ordinary skill in the art with the benefit of this disclosure.

If the controller 116 determines that the person 110 is close enough (e.g., horizontally within 1 foot) to the lighting device 102 for the VOC sensor unit 148 to reliably sense in the air one or more sweat compounds from the sweat produced by the person 110 (e.g., sweat produced by the apocrine glands of the person 110), the controller 116 may use sweat data just received from the VOC sensor unit 148. Alternatively, if the controller 116 determines that the person 110 is close enough to the lighting device 102 for the VOC sensor unit 148 to reliably sense in the air one or more sweat compounds from the sweat produced by the person 110 (e.g., sweat produced by the apocrine glands of the person 110), the controller 116 may instruct the VOC sensor unit 148 to sense sweat compound(s) and use the sweat data received from the VOC sensor unit 148 to extract or determine one or more parameters (e.g., the amount or concentration of a compound, whether the person 110 is dehydrated, etc.). The controller 116 may adjust the spectral distribution and/or the spatial distribution of the light provided by the lighting devices 102, 104 based on the one or more parameters in the manner described above with respect to parameters obtained from the sensor 112 attached to the person 110.

In some example embodiments, if the controller 116 determines that the person 110 is close (e.g., horizontally within 1 foot) to the lighting device 102 based on information from the location sensor unit 144, the controller 116 may control the light module 114 to emit, for example, an infrared light to cause the person 110 sweats more. Because of the increased likelihood that more sweat components from the sweat produced by the person 110 are airborne as the person 110 sweats more, the controller 116 may extract or otherwise determine one or more parameters from the sweat data received from the VOC sensor unit 148 and adjust the spectral distribution and/or the spatial distribution of the light provided by the lighting devices 102, 104 based on the one or more parameters in the manner described above with respect to parameters obtained from the sensor 112 attached to the person 110.

In some example embodiments, the controller 116 may execute a machine learning (ML) algorithm to determine the reliability of the sweat data received from the VOC sensor unit 148. The controller 116 may also execute the ML algorithm to determine the physiological and psychological state of the person 110 based on the sweat data received from the VOC sensor unit 148 and adjust the spectral distribution and/or the spatial distribution of the light provided by the lighting devices 102, 104 in the manner described above.

In some example embodiments, the controller 134 of the lighting device 104 may operate in the manner described with respect to the controller 116 of the lighting device 102 to control the light provided by the lighting device 104. The controller 134 of the lighting device 104 may also operate in the manner described with respect to the controller 116 of the lighting device 102 to control the light provided by the lighting device 102. For example, the lighting device 104 may operate as a master lighting device, where the controller 134 controls operations of other lighting devices including the lighting device 102 in the lighting system 100 to control the light provided by the lighting system 100. To illustrate, the controller 134 of the lighting device 102 may control the light provided by the lighting device 104 as well as the light provided by the lighting device 102 based on one or more parameters extracted and/or determined from sensor data received from the sensor 112 and/or the integrated sensors 118, 136. For example, the controller 134 may transmit a lighting control command generated based on the one or more parameters to the lighting device 102, and the controller 116 of the lighting device 102 may receive the lighting control command and control the light provided by the light module 114 of the lighting device 102 accordingly.

In some example embodiments, the network device 106 may be a server that performs the functions described herein with respect to the controller 116 of the lighting device 102. For example, the network device 106 may include a controller 142 that can receive sensor data from the sensor 112 and/or from the integrated sensor 118 and process the sensor data. The controller 142 may transmit light control commands to the lighting devices 102, 104 to control the lights provided by the lighting devices 102, 104 based on one or more parameters extracted or determined by the controller 142 from the sensor data.

By using sensor data that includes information about sweat produced by the person 110, the lighting system 100 can determine information about the physiological and/or psychological state of the person 110. Based on the sensor data obtained from the sensor 112, the lighting system 100 can adjust the light provided by the lighting system 100 (e.g., light provided by the lighting device 102, the lighting device 104, and/or another lighting device of the lighting system 100) to help improve the physiological and psychological state of the person 110.

In some alternative embodiments, the sensor 112 may be at a different location on the person 110 without departing from the scope of this disclosure. For example, the sensor 112 may include sensors that are at different locations on the person 110. In some example embodiments, the integrated sensors 118,136 may be omitted without departing from the scope of this disclosure. In some example embodiments, the person 110 may be at a different location in the area 108 than shown without departing from the scope of this disclosure. In some example embodiments, the lighting system 100 may include more or fewer lighting devices than shown without departing from the scope of this disclosure.

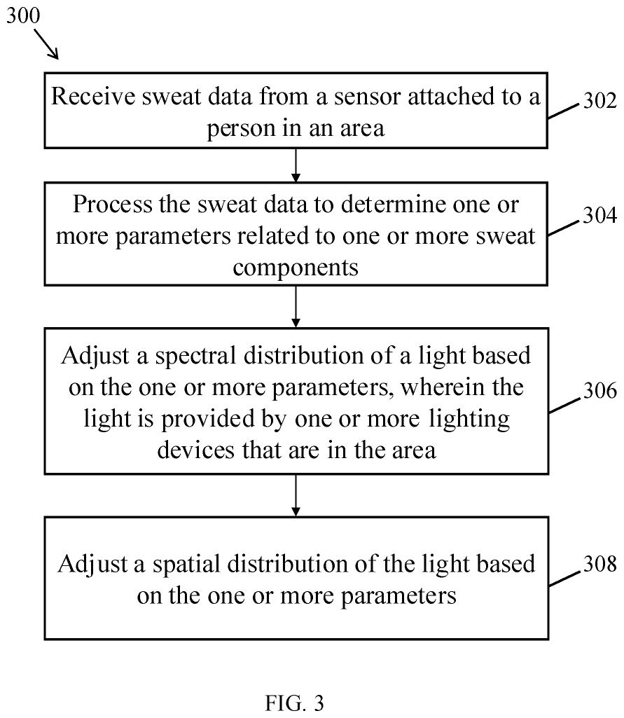

FIG. 3 illustrates a method 300 of lighting control based on sweat data from a sensor 112 according to an example embodiment. Referring to FIGS. 1-3, in some example embodiments, the method 300 include, at step 302, receiving sweat data from the sensor 112 attached to the person 110 in the area 108. For example, the controller 116 of the lighting device 102, the controller 134 of the lighting device 104, and/or the controller 142 of the network device 106 may receive the sensor data. The sweat data may include information related to sweat produced by the person 110. At step 304, the method 300 may include processing the sweat data to determine one or more parameters related to one or more sweat components. For example, a sweat component may be sodium, potassium, chloride, glucose, lactate, cortisol, a fatty acid, a protein, etc. The controller 116 of the lighting device 102, the controller 134 of the lighting device 104, and/or the controller 142 of the network device 106 may process the sensor data to determine parameters related to the sweat components. For example, the one or more parameters may include the amount, concentration, and/or ratio of analytes in the sweat produced by the person 110 as indicated or otherwise determined from the sweat data.

In some example embodiments, at step 306, the method 300 may include adjusting a spectral distribution 202 of the light based on the one or more parameters. The light is provided by one or more lighting devices 102, 104 that are in the area 108. The controller 116 of the lighting device 102, the controller 134 of the lighting device 104, or the controller 142 of the network device 106 may adjust the spectral distribution 202 of the light based on the one or more parameters. For example, the controller 116 of the lighting device 102 may provide lighting control commands to the light module 114 of the lighting device 102, for example, to start emitting or change the intensity of a light (e.g., a cyan light, an infrared light, a UV light) having a wavelength in a particular wavelength range. The light module 114 of the lighting device 102 may adjust the light provided by the lighting device 102 in response to the lighting control commands from the controller 116. The controller 116 of the lighting device 102 may provide lighting control commands to the light module 114 of the lighting device 102, for example, to start emitting or change the intensity of a light (e.g., yellow light) having a wavelength in a particular wavelength range such that the change in the CCT of the light provided by the lighting device 102 is limited to a particular range (e.g., 300K).

In some example embodiments, as described above, one or more sweat components of the sweat produced by the person 110 may include glucose. The spectral distribution 202 of the light provided by the lighting device 102 may be adjusted by introducing a cyan light or by increasing an intensity of the cyan light included in the light provided by the lighting device 102 in response to determining that an amount of the glucose is below a glucose threshold. For example, the glucose threshold can be set to a desired value that is specific to the person 110 or that is recommended for the general population, for example, by a governmental or private health entity. The spectral distribution of the light provided by the lighting device 104 may also be adjusted in the same manner as described with respect to the spectral distribution 202 of the light provided by the lighting device 102.

In some example embodiments, as described above, one or more sweat components of the sweat produced by the person 110 may include lactate. The spectral distribution 202 of the light provided by the lighting device 102 may be adjusted by introducing an infrared light to the light or by increasing an intensity of the infrared light included in the light provided by the lighting device 102 in response to determining that an amount or concentration of the lactate exceeds a lactate threshold. For example, the lactate threshold can be set to a desired value that is specific to the person 110 or that is recommended for the general population, for example, by a governmental or private health entity. The spectral distribution of the light provided by the lighting device 104 may also be adjusted in the same manner as described with respect to the spectral distribution 202 of the light provided by the lighting device 102.

In some example embodiments, as described above, one or more sweat components of the sweat produced by the person 110 may include cortisol. The spectral distribution 202 of the light provided by the lighting device 102 may be adjusted by introducing a cyan light or by increasing an intensity of the cyan light in response to determining that an amount or concentration of the cortisol is below a cortisol threshold. For example, the cortisol threshold can be set to a desired value that is specific to the person 110 or that is recommended for the general population, for example, by a governmental or private health entity. The spectral distribution of the light provided by the lighting device 104 may also be adjusted in the same manner as described with respect to the spectral distribution 202 of the light provided by the lighting device 102.

In some example embodiments, as described above, one or more sweat components of the sweat include sodium, potassium, and chloride. The spectral distribution 202 of the light provided by the lighting device 102 may be adjusted in response to determining that the person 110 is dehydrated at least based on an amount or concentration of one or more of the sodium, the potassium, and the chloride. The spectral distribution of the light provided by the lighting device 104 may also be adjusted in the same manner as described with respect to the spectral distribution 202 of the light provided by the lighting device 102.

In some example embodiments, the spectral distribution 202 of the light provided by the lighting device 102 is adjusted further based on sensor data received from the sensor 112 indicating the temperature of the person 110, the heart rate of the person 110, the blood pressure of the person 110, and/or the respiratory rate of the person 110. For example, the controller 116 of the lighting device 102 may use the temperature and/or heart rate information in addition to one or more parameters determined from the sweat data (e.g., concentration of sodium in the sweat, concentration of another analyte in the sweat, and/or ratio of a couple of analytes in the sweat) to determine whether the person 110 is dehydrated and to adjust the spectral distribution 202 of the light provided by the lighting device 102. The spectral distribution of the light provided by the lighting device 104 may also be adjusted in the same manner as described with respect to the spectral distribution 202 of the light provided by the lighting device 102.

In some example embodiments, the controller 116 of the lighting device 102 may determine whether the sweat produced by the person 110 is regular sweat or stress sweat based on the one or more parameters determined from the sweat data. In response to determining that the sweat is stress sweat, the controller 116 of the lighting device 102 may adjust the light provided by the lighting device 102 by controlling the light module 114 of the lighting device 102 to reduce an intensity of a cyan light included in the light provided by the light module 114. For example, the controller 116 of the lighting device 102 can adjust the light (e.g., adjust the spatial distribution, adjust the spectral distribution) provided by the lighting device 102 to reduce a stress level of the person 110. The spectral distribution of the light provided by the lighting device 104 may also be adjusted in the same manner as described with respect to the spectral distribution 202 of the light provided by the lighting device 102.

In some example embodiments, the controller 116 of the lighting device 102 may transmit lighting control commands to the lighting device 104 to adjust the light provided by the lighting device 104. The controller 116 may generate the lighting control commands based on one or more parameters determined from the sweat data received from the sensor 112. The lighting device 104 may adjust the light provided by the lighting device 104 in response to the lighting control commands from the lighting device 102. Alternatively, the controller 142 of the network device 106 may transmit light control commands to one or more of the lighting devices of the lighting system 100, including the lighting devices 102, 104, to adjust the light provided by the lighting system 100 in the same manner as described with respect to the controller 116 of the lighting device 102. In general, adjustment of the light provided by one or more of the lighting devices 102. 104 results in a corresponding adjustment of the light provided by the lighting system 100.

In some example embodiments, at step 308, the method 300 may include adjusting a spatial distribution of the light emitted by the lighting device 102 based on the one or more parameters. For example, the controller 116 of the lighting device 102 may control the spatial distribution of the light provided by the lighting device 102 and the light provided by the lighting device 104 based on one or more parameters extracted and/or determined from sensor data received from the sensor 112. To illustrate, the controller 116 may adjust the spatial distribution of the light provided by the lighting device 102 instead of or in addition to changing the spectral distribution of the light. For example, in response to determining that the person 110 is dehydrated as described above, the controller 116 may control the light module 114 to change the spatial distribution of the light provided by the lighting device 102 from a first spatial distribution 138 to a second spatial distribution 140 shown in FIG. 1 or to another spatial distribution. The controller 116 may control transmit lighting control commands to the lighting device 104 to change the spatial distribution of the light provided by the lighting device 104.

In some alternative embodiments, the method 300 may include more or fewer steps than shown without departing from the scope of this disclosure. For example, one of the steps of the method 300 may be omitted without departing from the scope of this disclosure. In some alternative embodiments, the steps of the method 300 may be performed in a different order than shown without departing from the scope of this disclosure. For example, the step 308 may be performed before the step 306.

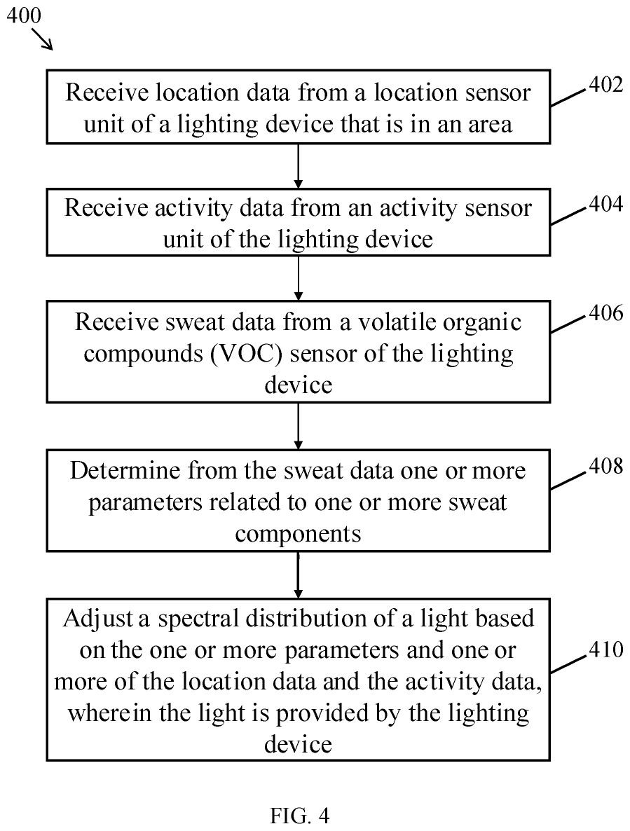

FIG. 4 illustrates a method of lighting control based on sweat data from a light fixture integrated sensor according to another example embodiment. Referring to FIGS. 1, 2, and 4, in some example embodiments, the method 400 includes, at step 402, receiving location data from the location sensor unit 144 of the lighting device 102 that is in the area 108. For example, the controller 116 of the lighting device 102 may receive the location data. A thermopile sensor, a passive infrared sensor, and/or another sensor may be used as the location sensor unit 144 to detect whether the area 108 is occupied and determine the location of the person 110 in the area 108.

In some example embodiments, at step 404, the method 400 includes receiving activity data from the activity sensor unit 146 of the lighting device 102. For example, the controller 116 of the lighting device 102 may receive the activity data. Microphones, a thermopile sensor, a humidity sensor, temperature sensor, and/or another sensor may operate individually or together as the activity sensor unit 146 to determine the activity of the person 110. For example, activity data from the activity sensor unit 146 may be used to determine whether the person 110 has just finished exercising based on the breathing rate of the person 110, sounds emitted by the person 110, etc. As another example, activity data from the activity sensor unit 146 may be used to determine whether the person 110 has just showered based on the humidity in the air and/or sounds associated with showering. As another example, activity data from the activity sensor unit 146 may be used to determine whether the person 110 has used a deodorant based on the sound associated with the spraying of a deodorant.

In some example embodiments, at step 406, the method 400 includes receiving sweat data from the VOC sensor unit 148 of the lighting device. The VOC sensor unit 148 may detect one or more sweat components in the air and transmit sweat data related to the sweat components. For example, the sweat data from the VOC sensor unit 148 may indicate or may be used to determine the amount or concentration of cortisol and/or other sweat components in sweat produced by the person 110. At step 408, the method 400 may include determining from the sweat data one or more parameters related to one or more sweat components. For example, the controller 116 may determine from the sweat data amount, concentration, ratio, etc. of analytes in the sweat produced by the person 110.

In some example embodiments, at step 410, the method 400 includes adjusting a spectral distribution 202 of the light provided by the lighting device 102 based on the one or more parameters and one or more of the location data and the activity data. To illustrate, before adjusting the light provided by the lighting device 102 based on the sweat data from the VOC sensor unit 148, the microprocessor 126 of the controller 116 may determine whether the sweat data is reliable based on location data from the location sensor unit 144 and/or the activity data from the activity sensor unit 146. For example, before processing and/or using the sweat data from the VOC sensor unit 148, the microprocessor 126 may determine whether the area 108 is occupied based on information from the location sensor unit 144. The controller 116 may adjust the light provided by the lighting device 102 based on the one or more parameters determined from the sweat data after determining that the area 108 is occupied. To illustrate, if the area 108 is occupied, the likelihood that the one or more compounds detected by the VOC sensor unit 148 are from sweat produced by the occupant (e.g., the person 110) is higher.

As another example, before processing and/or using the sweat data from the VOC sensor unit 148, the microprocessor 126 may determine from the location data from the location sensor unit 144 whether the person is close enough (e.g., horizontally within 1 foot) to the lighting device 102 for the VOC sensor unit 148 to reliably sense in the air one or more sweat compounds from the sweat produced by the person 110. The controller 116 may adjust the light provided by the lighting device 102 based on the one or more parameters determined from the sweat data after determining that the person 110 is within a threshold distance (e.g., 1 foot, 2 feet) of the lighting device 102.

As another example, before processing and/or using the sweat data from the VOC sensor unit 148, the microprocessor 126 may determine whether the person 110 just finished exercising based on information from the activity sensor unit 146 (e.g., from a thermopile sensor). To illustrate, if the person 110 just finished exercising (e.g., as determined based on heart rate and/or breathing rate), the likelihood that the one or more compounds detected by the VOC sensor unit 148 are from sweat produced by the person 110 is higher. The controller 116 may adjust the light provided by the lighting device 102 based on the one or more parameters determined from the sweat data after determining that the person 110 just finished exercising.

As yet another example, before processing and/or using the sweat data from the VOC sensor unit 148, the microprocessor 126 may determine whether the person 110 just showered and has not used a deodorant (which can affect the reliability of sensing by the VOC sensor unit 148) based on information from the activity sensor unit 146 (e.g., from a humidity sensor and microphones). To illustrate, if the person 110 just finished showering and has not yet used a deodorant, the likelihood that one or more sweat components detected by the VOC sensor unit 148 are from sweat produced by the person 110 instead of from the deodorant is relatively higher. The controller 116 may adjust the light provided by the lighting device 102 based on the one or more parameters determined from the sweat data after determining that the person 110 just finished exercising and did not apply a spray deodorant that can lead to unreliable detection by the VOC sensor unit 148.

In some example embodiments, the controller 116 of the lighting device 102 may transmit light control commands to the lighting device 104 in the manner described above to control the light provided by the lighting device 104 based on the one or more parameters determined from the sweat data and based on the location and/or the activity data. In some example embodiments, the controller 116 of the lighting device 102 may control the spatial distribution of the light provided by the lighting system 100 (i.e., the light provided by the lighting device 102, the light provided by the lighting device 104, and/or the light provided by another lighting device of the lighting system 100) based on the one or more parameters determined from the sweat data and based on the location and/or the activity data.

In some alternative embodiments, the method 400 may include more or fewer steps than shown without departing from the scope of this disclosure. In some alternative embodiments, the steps of the method 400 may be performed in a different order than shown without departing from the scope of this disclosure.

Although particular embodiments have been described herein in detail, the descriptions are by way of example. The features of the example embodiments described herein are representative and, in alternative embodiments, certain features, elements, and/or steps may be added or omitted. Additionally, modifications to aspects of the example embodiments described herein may be made by those skilled in the art without departing from the scope of the following claims, the scope of which are to be accorded the broadest interpretation so as to encompass modifications and equivalent structures.

1. A lighting control method comprising:
receiving, by a controller, sweat data from a sensor that is in proximity of a person in an area, wherein the sensor is configured to sense sweat produced by the person;
processing, by the controller, the sweat data to determine one or more parameters related to one or more sweat components of the sweat; and
adjusting, by the controller, a spectral distribution of an illumination light at least based on the one or more parameters, wherein the illumination light is provided by one or more lighting devices that are in the area, wherein the one or more sweat components of the sweat include one or more of glucose, lactate, cortisol, sodium, potassium, and chloride, wherein the one or more parameters include one or more amounts or one or more concentrations of the one or more sweat components of the sweat, and wherein the spectral distribution (202) of the illumination light is adjusted based on one or more amounts or one or more concentrations of the one or more sweat components of the sweat.

2. The lighting control method of claim 1, further comprising adjusting a spatial distribution of the illumination light based on the one or more parameters.

3. The lighting control method of claim 1, wherein the correlated color temperature (CCT) of the illumination light is maintained within a CCT range such that a change in the CCT of the illumination light resulting from adjusting the spectral distribution of the illumination light is imperceptible by the person.

4. The lighting control method of claim 1, wherein the sweat is sensed by the sensor continuously or at regular intervals and wherein the spectral distribution is adjusted continuously or at the regular intervals.

5. The lighting control method of claim 1, wherein the illumination light is a white light having a correlated color temperature in a range from 1800K to 6500K and a color rendering index of at least 70 and wherein the spectral distribution of the illumination light is adjusted by introducing to the illumination light one or more lights from among a violet light, a cyan light, a red light, a blue light, a yellow light, a near-infrared light, an infrared light, and an ultraviolet light, by adjusting one or more intensities of one or more of the violet light, the cyan light, the red light, the blue light, the yellow light, the near-infrared light, the infrared light, and the ultraviolet light included in the illumination light, or both.

6. The lighting control method of claim 1, wherein the one or more sweat components of the sweat include glucose and wherein the spectral distribution of the illumination light is adjusted by introducing a cyan light or by increasing an intensity of the cyan light included in the illumination light in response to determining that an amount of the glucose is below a glucose threshold.

7. The lighting control method of claim 1, wherein the one or more sweat components of the sweat include lactate and wherein the spectral distribution of the illumination light is adjusted by introducing an infrared light or near-infrared light to the illumination light or by increasing an intensity of the infrared light or near-infrared light included in the illumination light in response to determining that an amount or concentration of the lactate exceeds a lactate threshold.

8. The lighting control method of claim 1, wherein the one or more sweat components of the sweat include cortisol, wherein the spectral distribution of the illumination light is adjusted by introducing a cyan light or by increasing an intensity of the cyan light in response to determining that an amount or concentration of the cortisol is below a cortisol threshold, and wherein the spectral distribution of the illumination light is adjusted by turning off or reducing the intensity of the cyan light in response to determining that the amount or concentration of the cortisol is above a high cortisol threshold that is associated with sleep apnea.

9. The lighting control method of claim 1, wherein the one or more sweat components of the sweat include sodium, potassium, and chloride and wherein the spectral distribution of the illumination light is adjusted in response to determining that the person is dehydrated or well hydrated at least based on an amount or concentration of one or more of the sodium, the potassium, and the chloride.

10. The lighting control method of claim 1, wherein the spectral distribution of the illumination light is adjusted further based on sensor data received from the sensor and wherein the sensor data indicates at least one or more of a temperature of the person, a heart rate of the person, a heart rate variability of the person, a blood pressure, and a respiratory rate of the person.

11. The lighting control method of claim 1, wherein the spectral distribution of the illumination light is adjusted by reducing an intensity of a cyan light included in the illumination light in response to determining that the sweat is stress sweat based on the one or more parameters.

12. A lighting control system comprising a controller configured to:
receive sweat data from a sensor that is in proximity of a person in an area, wherein the sensor is configured to sense sweat produced by the person;
process the sweat data to determine one or more parameters related to one or more sweat components of the sweat; and
adjust a spectral distribution of an illumination light at least based on the one or more parameters, wherein the illumination light is provided by one or more lighting devices that are in the area, wherein the one or more sweat components of the sweat include one or more of glucose, lactate, cortisol, sodium, potassium, and chloride, wherein the one or more parameters include one or more amounts or one or more concentrations of the one or more sweat components of the sweat, and wherein the spectral distribution of the illumination light is adjusted based on one or more amounts or one or more concentrations of the one or more sweat components of the sweat.

13. The lighting control system of claim 12, wherein the controller is further configured to adjust a spatial distribution of the illumination light based on the one or more parameters.

14. The lighting control system of claim 12, wherein the correlated color temperature (CCT) of the illumination light is maintained within a CCT range such that a change in the CCT of the illumination light resulting from adjusting the spectral distribution of the illumination light is imperceptible by the person.
